SEQUENCE LISTING (3 sequences)

SEQ ID NO: 1 - protein, 569 aa, Pseudomonas chlororaphis
MNSSHSGFEETQGTRSFPNIDLLYDYGQFLRRATQAIGHLPPAIADKHIGIVGAGLSGLIAAYELLRAGATRITLFEASKDHLGGRFLTQCFDETQPQFIAEMGAMRFPPSEVGLFHYLDRFGIGTTEAFPDPGVVDTEIHYRGETHHWPAGENPPPIFNTVHRGWNAFLKEGVRLDDGTFLAPPLLLTELLEKQQYAEVQPEWQRYLDCFGDHSFYSAMVQIFTSKNPPGGKRWRKPEDFHLFGSLGIGSGGFQSVYRASFTEILRLVINAFEVNQRLVPNGISTLWERIADEEFHGIALRDRICRTRIESISKGADGQILLQGCNGETYGCDRVITTATTRALQVNMRLTQAEQFVNRDVARAINETHMVGSSKLFILTRDKFWLKHGLAQNIQTDTLVKGVYCLDYAPQDPDSWGVVLISYTWEDDSHKMVSMTDKVQRCKRLVDELAMSSPEFARHLVPLDGDYQRYVLEYDWLTDKQALGAFKLNFPGDDLYSQRLFYQFKTALDPQQDTGLYLAGCGSSFTGGWAEGAVQTALNGACAVIASLGGELLEGNPLFDLQSHFRYC

SEQ ID NO: 2 - protein, 447 aa, Pseudomonas chlororaphis O6
MYSQLSIEALVDGFRQGKLCLDSWHRTLLEQYADAGHLNSFISFAPQAFSGDTGTPEQRQLPLYGIPVSFKDNINASGLPTTAGTPGLADYQPAADAGIVQRFKALGARVAGKNNMHELSFGVTSANLSYGAVVNPRHLQHSAGGSSGGCAAAVAAGLVPCAVGTDTGGSVRIPAAFCGVVGFRPSTGVYPGDGIVPVSQTKDSPGLLTRSLEDALFVHRQLTGASCAAPIEPKRLRIGIPQQFFWSELETKVHSDCRAAIDCLADQGVEMVPVDDRLIGEINESIQFPLPIFEFFIDFPRFLLKAGCGERFLEILAQIRDPQIKKTLMAQLESPAISYEDYVQALMSKIRLDREYAGLFQQHRLDLIAYPTVSCTAPLLGGSGAQEHFERFVRNTDPASNLAAPSVSVPVAAAGSLPVGLSFDALPGQDHQLLQMAQQLSRLLALR

SEQ ID NO: 3 - protein, 425 aa, Pseudomonas chlororaphis O6
MRGTSIASSPSTSHRTVAERRPSVIGGAMIIGGTIVGAGMFSLPVMMSGLWFQGSLAVLVLSWFCTLHSGLMILEANLNYRPGASFSTITRDLIGRRWSLFNGATLVFVLYILTYAYISASGSVIRHTAHTLGLEISSRNGGLLFALAVALVVWLSTAAVSRITTLVFGAKILAFFLTFGGLLGHVQPQVLLDLGNPQPDYLPYLFMTLPFCLTSFGFHGNVPSLMKHYGKDPQRIRNCLLIGTLIALVLYTVWMLCTMGTLPRDQFKDIARQGGTIDVLISALGGMLNSARVDLLLTLFSNFAVACSFLGVTLGLFDYIADSLGFDDSPRGRLKSALVTFAPPIGCALIWPEGFILAIGFAGLAATVWAVITPALLAHASRKRFASPLYRVWGGTPMIVLVLLFGLANALAHLLFSFDLLPVWR

(19) United States
(12) Patent Application Publication
(10) Pub. No.: US 2023/0062510 A1
(43) Pub. Date: Mar. 2, 2023

(54) METHOD TO CONSTRUCT EFFICIENT INDOLE-3-ACETIC ACID-PRODUCING MICROBES

(71) Applicant: Gary Zhan, North Logan, UT (US)

(72) Inventor: Gary Zhan, North Logan, UT (US)

(21) Appl. No.: 17/411,056

(22) Filed: Aug. 25, 2021

(51) Int. Cl. C12N 15/78; C12P 17/10

(52) U.S. Cl. CPC C12N 15/78; C12P 17/10

(57) ABSTRACT

The present invention comprises a novel method to engineer microbes to efficiently produce the plant auxin indole-3-acetic acid. While some microorganisms including soil bacteria are known to produce indole-3-acetic acid, the yields are often very low. This technology allows the engineering of selected microbes for a strong ability to produce indole-3-acetic acid. Specifically, indole-3-acetic acid biosynthetic genes and a tryptophan transporter are expressed in a microbial host to efficiently convert tryptophan into indole-3-acetic acid. The engineered strains can be used in industry to produce this plant auxin or directly applied in agriculture to promote plant growth.

### REFERENCE TO A “SEQUENCE LISTING”

The amino acid sequence of a representative tryptophan 2-monooxygenase, Pc-IAA1, is shown in sequence listing.

The amino acid sequence of a representative indoleacetamide hydrolase, Pc-IAA2, is shown in sequence listing.

The amino acid sequence of a representative tryptophan transporter, Pc-IAA3, is shown in sequence listing.

### BACKGROUND

The number of hungry people in the world has been increasing since 2014, and about 821 million were malnourished in 2017. By 2050, the human population is projected to surpass 9.5 billion, an increase of nearly 2 billion. A combination of technologies will be needed to produce sufficient quality foods to sustain this projected population. Increasing agricultural output is challenged by greater extremes of climate variability and marginalized agricultural soils due to urbanization and competition for water. Additional pressures include overuse of fertilizers and pesticides, which contribute to environmental pollution, eutrophication induced “dead-zones” in lakes and oceans, as well as adverse health effects, both short-term and long-term, to humans [1].

Microbes that promote plant health are a promising tool to sustainably boost soil fertility and crop production [2], with roles in the development and maintenance of soil structure, nutrient cycling, and a variety of interactions with plant roots [3].

Soil bacteria are known to benefit crops in a variety of ways, including releasing the auxin indole-3-acetic acid (IAA). IAA is the most common naturally occurring plant hormone, which regulates various aspects of plant growth and development, such as cell division and elongation, tissue differentiation, apical dominance, as well as responses to light exposure and pathogens [4]. In addition to the growth-promoting properties, IAA can also inhibit weed (Cyperus rotundus L.) growth at a concentration of 50 mg/L. It has the potential to be used as an herbicidal bioproduct to replace the chemical herbicides [5]. However, the yield of IAA in soil bacteria is typically too low to have a significant impact on crop growth. For example, Pseudomonas chlororaphis O6 is a crop-benefiting soil bacterium and a natural IAA producer. The highest production level of IAA by this strain in flasks under optimal culture conditions was reported to be lower than 10 mg/L [6]. It is expected that the actual production of IAA in soil would be much lower due to limited nutrients and relatively poor growth conditions. Therefore, to improve the utility of soil bacteria in agriculture, it is critical to enable them to produce IAA in higher yields. Furthermore, efficient IAA-producing microbial strains will enable industrial production of this valuable agricultural natural product.

### BRIEF DESCRIPTION OF THE DRAWINGS

FIG. 1 illustrates the subsystem analysis of the coding sequences in Pseudomonas chlororaphis O6.

FIG. 2 shows two representative IAA biosynthetic pathways in bacteria.

FIG. 3 shows the putative IAA biosynthetic gene cluster in Pseudomonas chlororaphis O6 and the predicted functions of the genes.

FIG. 4 shows the subsystem analysis of the coding sequences in Pseudomonas putida KT2440.

FIG. 5 illustrates the construction of an expression plasmid harboring Pc-iaa1 and Pc-iaa2.

FIG. 6 demonstrates the analysis and characterization of IAA production by Pseudomonas putida KT2440/pGZ6.

FIG. 7 shows the plasmid map of pGZ5S.

### DETAILED DESCRIPTION

The present disclosure covers methods for engineering microorganisms for the capability to efficiently produce plant auxins. In the following description, numerous specific details are provided for a thorough understanding of specific preferred embodiments. However, those skilled in the art will recognize that embodiments can be practiced without one or more of the specific details, or with other methods, components, materials, etc. In some cases, well-known structures, materials, or operations are not shown or described in detail in order to avoid obscuring aspects of the preferred embodiments. Furthermore, the described features, genes, structures, strains, or characteristics may be combined in any suitable manner in a variety of alternative embodiments. Thus, the following more detailed description of the embodiments of the present invention, as illustrated in some aspects in the drawings, is not intended to limit the scope of the invention, but is merely representative of the various embodiments of the invention.

In this specification and the claims that follow, singular forms such as “a,” “an,” and “the” include plural forms unless the content clearly dictates otherwise. All ranges disclosed herein include, unless specifically indicated, all endpoints and intermediate values. In addition, “optional” or “optionally” refer, for example, to instances in which subsequently described circumstance may or may not occur, and include instances in which the circumstance occurs and instances in which the circumstance does not occur. The terms “one or more” and “at least one” refer, for example, to instances in which one of the subsequently described circumstances occurs, and to instances in which more than one of the subsequently described circumstances occurs.

In one embodiment, the present disclosure provides methods for engineering microorganisms including soil bacteria to produce the plant auxin IAA. By way of example, the present disclosure provides for overexpressing two IAA biosynthetic enzymes from Pseudomonas chlororaphis O6 in Pseudomonas putida KT2440 to create IAA in a high yield. Furthermore, a tryptophan transporter is introduced into the system to facilitate the transport of the substrate and subsequently the production of IAA. The methods described herein generally provide for a method to enable soil bacteria to efficiently produce IAA to better promote crop growth. Preferably, this method allows soil bacteria to produce IAA by incorporating IAA biosynthetic enzymes and an efficient tryptophan transporter, which will make soil bacteria more beneficial to crops in agriculture and useful strains for industrial production of IAA. The details for the procedure are provided.

The present disclosure also provides for the methods to express useful enzymes and substrate transporters in soil bacteria.

The genes encoding the IAA biosynthetic genes and tryptophan transporter can be directly amplified from the genome or cDNA of a related microbial strain or chemically synthesized. The genes may be modified for improved activity or expression.

Expression of the IAA biosynthetic genes and tryptophan transporter can be achieved in a variety of hosts such as Escherichia coli, Pseudomonas putida or other microbial strains. Any suitable bacterial strain, vector or culture condition may be used for the expression of the IAA biosynthetic genes and tryptophan transporter and production of IAA. By way of example, suitable bacterial strains include Pseudomonas putida . Alternatively, any species or strain of Pseudomonas may be used. Broadly, a suitable microbial strain is any microbial strain capable of expressing IAA biosynthetic genes and/or tryptophan transporter. In some embodiments, IAA may be generated by a microbial strain harboring a vector or vectors that encode for IAA biosynthetic genes and tryptophan transporter. The vector or vectors may be plasm ids.

IAA may be produced by microbes harboring IAA biosynthetic genes from exogenously supplied substrates such as L-tryptophan. IAA may also be generated by microbes harboring IAA biosynthetic genes directly without supply of any said substrates.

IAA may be produced by microbes harboring IAA biosynthetic genes and tryptophan transporter from exogenously supplied substrates such as L-tryptophan. IAA may also be generated by microbes harboring IAA biosynthetic genes and tryptophan transporter directly without supply of any said substrates.

The engineered microbial strains that harbor IAA biosynthetic genes and tryptophan transporter are grown in an appropriate medium. If there is an inducible promoter in the vector, a specific inducer will be added into the culture to induce protein expression. If a constitutive promoter is used, no inducer is needed.

The following examples are illustrative only and are not intended to limit the disclosure in any way. One skilled in the art would recognize various known methods and conditions for cloning or synthesizing IAA biosynthetic genes and tryptophan transporter, expressing IAA biosynthetic genes and tryptophan transporter in a soil bacterium, and analyzing the production of IAA. Each of these various embodiments are within the scope of the invention.

### EXAMPLES

The following materials and methods may be used in carrying out the various embodiments of the invention.

### Example 1. Bacterial Strains, Vectors, and Culture Conditions

Pseudomonas chlororaphis O6 is a crop-benefiting soil bacterium discovered in northern Utah. Escherichia coli XL1-Blue (Agilent) was used for general cloning. Pseudomonas putida KT2440 (ATCC 47054) was acquired from the American Type Culture Collection (ATCC) and was used for heterologous expression of the auxin biosynthetic enzymes and tryptophan transporter.

Escherichia coli strains were routinely grown at 37° C. Pseudomonas chlororaphis O6 and Pseudomonas putida KT2440 were grown at 28° C. Lysogeny Broth (LB) with 2% agar plates were used for general cultivation of microbial strains. LB medium contains 10 g/L tryptone (Fisher Scientific, Hampton, N.H., USA), 5 g/L yeast extract (Fisher Scientific, Hampton, N.H., USA), and 10 g/L sodium chloride in distilled water.

### Example 2. Genome Analysis of Pseudomonas chlororaphis O6 and Pseudomonas putida KT2440

The genome sequences of Pseudomonas chlororaphis O6 and Pseudomonas putida KT2440 were downloaded from GenBank. The sequences were then analyzed through the Rapid Annotation using Subsystem Technology (RAST) [7]. The functions of the genes in the putative IAA biosynthetic gene cluster were predicted using NCBI BLAST.

### Example 3. DNA Manipulations

The genomic DNA of Pseudomonas chlororaphis O6 was isolated using standard methods. Plasmids in Escherichia coli were extracted using a GeneJET™ Plasmid Miniprep Kit (Thermo Fisher Scientific).

### Example 4. Plasmid Construction

The 3,090-bp Pc-iaa1-Pc-iaa2 gene fragment was amplified from the genome of Pseudomonas chlororaphis O6 via PCR using primers Pc-IAA1-Pmel-Ndel-F (5′-AATTGTTTAAACCATATGAACTCCTCCCACTCCGGTTTC-3′) and Pc-IAA2-HindIII-R (5′-AATTAAGCTTTCAGCGCAGGGCCAGCAGG-3′). Similarly, the 4,465-bp Pc-iaa1-Pc-iaa2-Pc-iaa3 gene fragment was amplified from the genome using primers Pc-IAA1-Pmel-Ndel-F and Pc-IAA3-Hindlll-R (5′-AATTAAGCTTCTAACGCCAGACCGGCAGCAG-3′). These PCR products were first ligated into pJET1.2 for sequencing. They were then ligated into pMES between Pmel and HindIII to yield pGZ6 and pGZ5S.

### Example 5. Production of IAA by Engineered Pseudomonas putida KT2440 Strains

pGZ6 and pGZ5S were introduced into Pseudomonas putida KT2440 through electroporation. The engineered strains were grown in 250-mL Erlenmeyer flasks containing 50 mL of LB medium supplemented with 50 μg/mL kanamycin at 28° C. and 250 rpm. After 2 hours, L-rhamnose (0.2%) was added as the inducer, together with 5 mg of L-tryptophan. The cultures were maintained at 28° C. and 250 rpm for an additional 2 days. The samples were then taken from the flasks for LC-MS analysis to detect the production of IAA.

To find out the effect of the amounts of substrate on IAA production, different concentrations (0, 0.4, 0.8, 1.6, 3.2, 6.4, and 12.8 g/L) of L-tryptophan were added into the induced broths. A standard curve of IAA was established based on the correlations of peak areas to amounts of standard IAA. The titers were quantified on HPLC according to the peak areas.

To test the effect of the antibiotic on the production of IAA, I also measured the production of IAA by the strains in the absence of kanamycin. Two different substrate concentrations, 0 and 0.8 g/L, were examined. All the titers were obtained from two independent experiments.

### Example 6. Discovery of a Putative IAA Biosynthetic Gene Cluster from the Genome of Pseudomonas chlororaphis O6

Pseudomonas chlororaphis O6 is a crop-benefiting soil microbe isolated from Utah and it is a natural IAA-producer [6]. However, how this strain produces IAA remains to be unveiled. The genome of this strain was analyzed to scan for the genes potentially involved in IAA biosynthesis in it. The genome sequence was downloaded from GenBank (accession no. GCF_000264555) [8] and analyzed through the Rapid Annotation using Subsystem Technology (RAST).

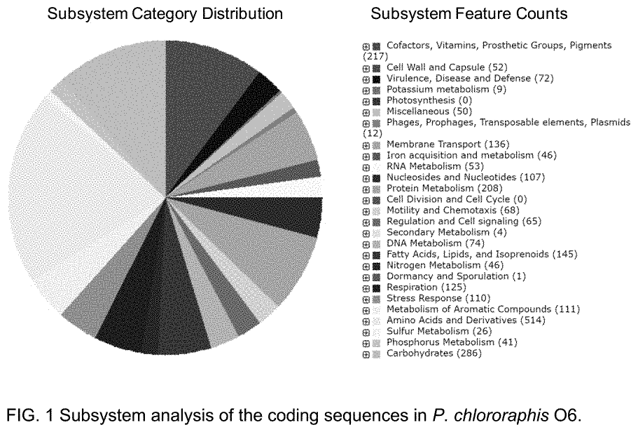

The whole genome contains 6.98 million base pairs, with 62.9% GC content. There are 6,414 coding sequences. The subsystem category distribution is shown in FIG. 1. The largest subsystem is “amino acids and derivatives”, followed by “carbohydrates,” “cofactors, vitamins, prosthetic groups, pigments,” and “protein metabolism.”

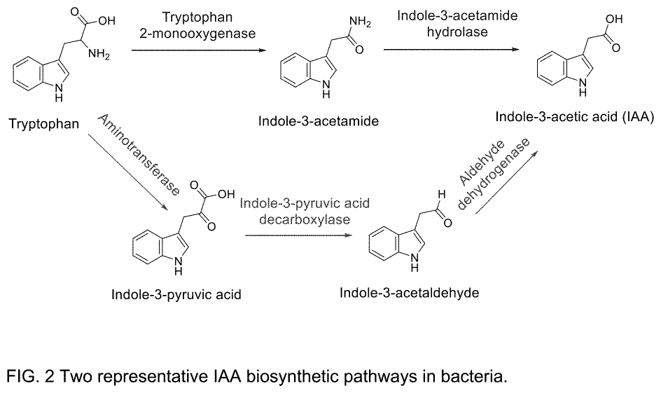

Previous studies reported that various routes are used by bacteria to synthesize IAA [9], and two representative pathways are shown in FIG. 2. Route 1 is catalyzed by two enzymes. The substrate tryptophan is converted to indole-3-acetamide by a tryptophan 2-monooxygenase. The amino group of this intermediate is then removed by an indole-3-acetamide hydrolase to yield IAA. In route 2, tryptophan is deaminated by an aminotransferase to yield indole-3-pyruvic acid, which is then converted to indole-3-acetaldehyde by a decarboxylase. The aldehyde group is then oxidized into carboxyl by an aldehyde dehydrogenase to generate IAA.

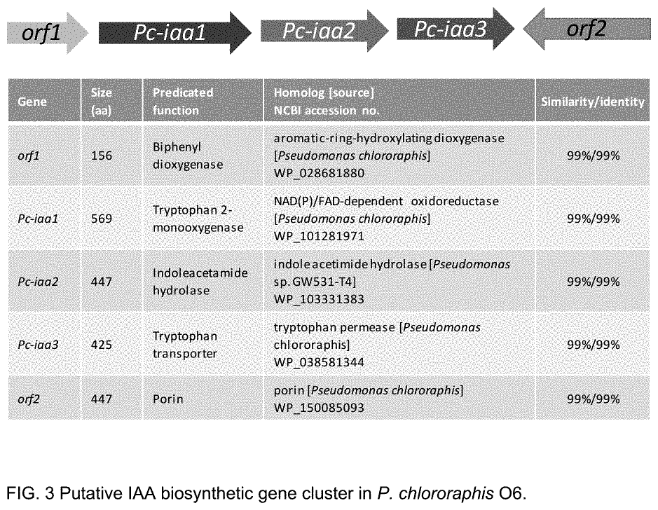

Within the subsystem of “amino acids and derivatives,” a gene cluster was discovered that contains three genes, including Pc-iaa1, Pc-iaa2 and Pc-iaa3 (FIG. 3). Based on the BLAST analysis, these three genes were predicted to be tryptophan 2-monooxygenase, indole-3-acetamide hydrolase, and tryptophan transporter, respectively (FIG. 3). Therefore, it is proposed that this gene cluster is responsible for the biosynthesis of IAA in Pseudomonas chlororaphis O6 via route 1 (FIG. 2). The gene cluster is flanked by two genes, orf1 and orf2, which don't seem to be involved in IAA biosynthesis based on the predicted functions.

### Example 6. Functional Characterization of the Putative IAA Biosynthetic Genes Through Heterologous Expression and IAA Production in Engineered Pseudomonas putida KT2440

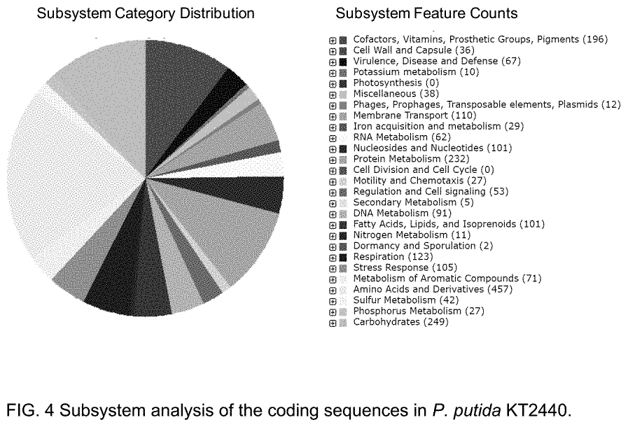

To characterize the functions of the three genes and create a highly efficient IAA-producing strain, Pseudomonas putida KT2440 was chosen as the host. This bacterial strain is a model soil bacterium and has been previously used as a host for heterologous DNA expression for different biotechnological purposes [10]. More importantly, Pseudomonas putida KT2440 lacks the ability to produce IAA. In fact, the genome of this strain (GenBank accession no. GCF_000007565) [11] was also analyzed through RAST. The size of this genome is 6.18 million base pairs, with 61.5% GC content. There are 5,715 coding sequences, falling into different subsystem categories based on their involvement in cellular metabolism (FIG. 4).

Similar to Pseudomonas chlororaphis O6, the largest subsystem in Pseudomonas putida KT2440 is “amino acids and derivatives,” followed by “carbohydrates,” “protein metabolism,” and “cofactors, vitamins, prosthetic groups, pigments.” As expected, no IAA biosynthetic genes were found in this strain, which provides an excellent “blank” strain for characterization of the genes from Pseudomonas chlororaphis O6.

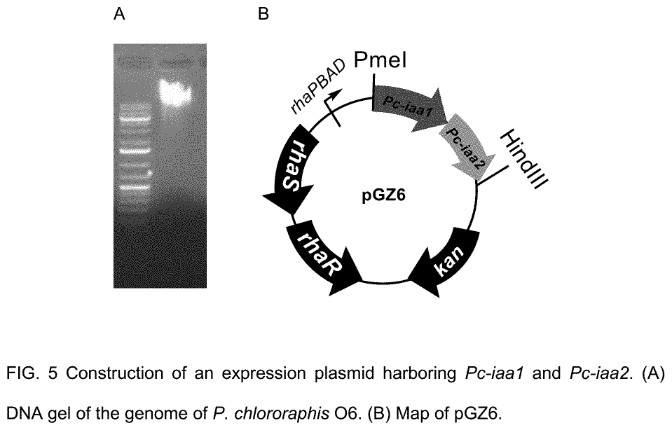

To this end, the two genes, Pc-iaa1 and Pc-iaa2, were amplified from the genome of Pseudomonas chlororaphis O6 (FIG. 5A). This PCR product was ligated into the cloning vector pJET1.2 and subsequently the expression vector pMES between Pmel and HindIII. The resulting plasmid pGZ6 (pMES-Pc-iaa1-Pc-iaa2) (FIG. 5B) was introduced into Pseudomonas putida KT2440 through electroporation. This engineered strain was grown in LB broth supplemented with kanamycin for product analysis, with L-rhamnose as the inducer and tryptophan as the substrate. Wild type Pseudomonas putida KT2440 was used as negative control.

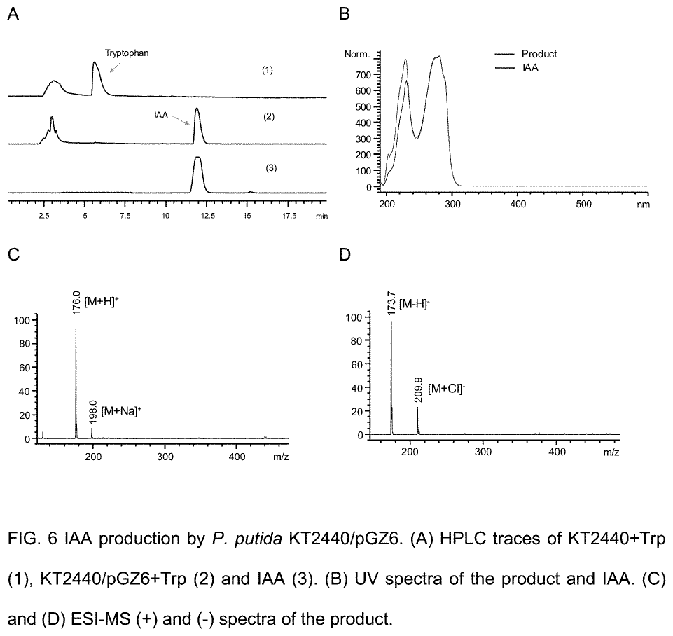

As shown in FIG. 6A, wild type Pseudomonas putida KT2440 did not produce any IAA and the substrate tryptophan was still in the broth (trace 1). By contrast, Pseudomonas putida KT2440/pGZ6 consumed all tryptophan and yielded a less polar product at 12 min (trace 2). This peak has the same retention time as the commercial standard of IAA (trace 3). A comparison of the UV spectra of this product with IAA indicated that they have identical UV absorptions (FIG. 6B). Furthermore, the ESI-MS spectra revealed that this product has a molecular weight of 175, according to the ion peaks [M+H]⁺ at m/z 176.0, [M+Na]⁺ at m/z 198.0, [M−H]⁻ at m/z 173.7, and [M+Cl]⁻ at m/z 209.9 shown in FIGS. 6C and 6D. Therefore, the biosynthesized product in the broth of Pseudomonas putida KT2440/pGZ6 was confirmed to be IAA. Since wild type Pseudomonas putida KT2440 does not generate IAA naturally, the production of this plant auxin in the engineered strain must be due to the heterologous expression of Pc-IAA1 and Pc-IAA2 from Pseudomonas chlororaphis O6. This work thus not only allowed the characterization of the two IAA biosynthetic genes in Pseudomonas chlororaphis O6, but also yielded an engineered strain of Pseudomonas putida capable of producing IAA.

### Example 7. Effect of the Substrate Concentration on IAA Production in Pseudomonas putida KT2440/pGZ6

As shown in FIG. 2, tryptophan is the substrate for IAA biosynthesis in bacteria. Availability of this molecule in the system would thus directly affect the amounts of IAA to be synthesized. The titers of IAA in Pseudomonas putida KT2440/pGZ6 were measured by supplementing various amounts of tryptophan into the fermentation broth. Seven concentrations of tryptophan were tested, including 0, 400, 800, 1600, 3200, 6400, and 12800 mg/L. Higher concentrations were not studied as the solubility of tryptophan in water is ˜11.4 g/L at room temperature. Two days after the substrate addition, the broths were subjected to HPLC analysis, and the amounts of IAA were calculated based on a standard curve of commercial IAA.

The measured titers of IAA in Pseudomonas putida KT2440/pGZ6 are listed in Table 1. When no tryptophan was added, Pseudomonas putida /pGZ6 produced IAA at 128.9 mg/L. This indicated that the bacterium can obtain tryptophan from the culture medium or synthesize this amino acid from the nutrients. The next step was to examine whether Pc-IAA1 and Pc-IAA2 have the ability to convert more tryptophan to IAA. To this end, tryptophan was supplied into the cultures at different concentrations, and found that the corresponding titers steadily increased until the substrate concentration reached 6.4 g/L (Table 1). When the substrate was supplied at 12.8 g/L, the titer dropped to 5.664 g/L. This was likely due to the inefficient substrate transport and potential substrate inhibition. The best titer of IAA was 6.218 g/L when tryptophan was supplemented at 6.4 g/L.

TABLE 1
Production titers of IAA in the engineered
Pseudomonas putida strains.

| | IAA titer (mg/L) | |
|---|---|---|
| Trp | Pseudomonas | Pseudomonas |
| (mg/L) | putida/pGZ6 | putida/pGZ5S |
| 0 | 128.9 ± 18.6 | 129.3 ± 0.8 |
| 400 | 522.2 ± 71.9 | 380.7 ± 47.7 |
| 800 | 1163.0 ± 169.1 | 969.5 ± 99.5 |
| 1600 | 1948.8 ± 243.0 | 1676.1 ± 172.2 |
| 3200 | 3895.0 ± 248.7 | 2859.3 ± 324.4 |
| 6400 | 6218.1 ± 35.7 | 6295.0 ± 967.5 |
| 12800 | 5664.0 ± 47.8 | 11657.9 ± 472.0 |

### Example 8. Enhanced Production of IAA Through the Introduction of a Tryptophan Transporter into Pseudomonas putida KT2440

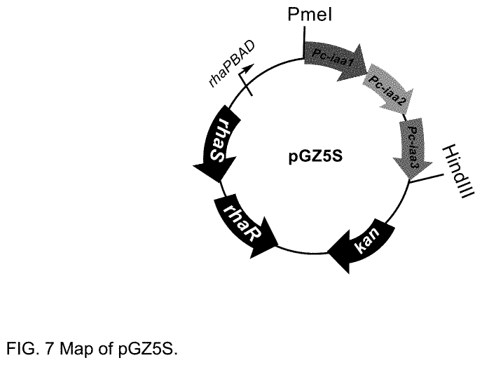

Besides Pc-iaa1 and Pc-iaa2, there is another gene, named Pc-iaa3, in the gene cluster. This gene was predicted to encode a tryptophan transporter or permease based on the BLAST analysis of its amino acid sequence (FIG. 3). It was hypothesized that adding this gene into the system may increase the uptake of tryptophan into the cells and thus enhance the production of IAA. The three genes were cloned from Pseudomonas chlororaphis O6 and ligated into pMES to yield pGZ5S (pMES-Pc-iaa1-Pc-iaa2-Pc-iaa3) (FIG. 7). The plasmid was introduced into Pseudomonas putida KT2440 and the production of IAA by this engineered strain was tested in the presence of different concentrations of tryptophan. As shown in Table 1, the titers of IAA in Pseudomonas putida KT2440/pGZ5S were similar to those in Pseudomonas putida KT2440/pGZ6 when tryptophan was supplied at up to 6.4 g/L. However, when the substrate concentration was increased to 12.8 g/L, the titer of IAA in Pseudomonas putida KT2440/pGZ5S reached 11.658 g/L after 2 days, much higher than that (5.664 g/L) in Pseudomonas putida KT2440/pGZ6. This indicated that the tryptophan transporter Pc-IAA3 made the transport of the substrate into the cells more efficient. Overall, these results confirmed that the addition of Pc-IAA3 significantly facilitated the transport of tryptophan into Pseudomonas putida and enhanced the production of IAA.

### Example 9. IAA Production by Engineered Pseudomonas putida KT2440 Strains in the Absence of Kanamycin

Antibiotics are commonly used as a selection pressure to maintain the plasmid stability in engineered strains. However, use of antibiotics can kill other beneficial soil microbes and contaminate the soil and crops, posing a threat to human health. Furthermore, the presence of an antibiotic in the fermentation broth will make the downstream purification steps during the industrial production of IAA more challenging. To construct and apply highly efficient IAA-producing soil bacteria for agricultural and industrial applications, the use of antibiotics should be avoided. It was thus important to test whether the engineered strains can still efficiently produce IAA in the absence of kanamycin. As shown in Table 2, both Pseudomonas putida KT2440/pGZ5S and Pseudomonas putida KT2440/pGZ6 produced IAA at a significant level. When no tryptophan was supplied into the medium, IAA was produced at 106.9 and 109.8 mg/L by the two strains, respectively. The titers in Pseudomonas putida KT2440/pGZ6 and Pseudomonas putida KT2440/pGZ5S reached 552.9 and 561.9 mg/L, respectively, in the presence of 800 mg/L tryptophan. These results revealed that in the absence of kanamycin, the two engineered strains can still generate IAA efficiently, which significantly improved the utility of these strains.

TABLE 2
Production of IAA by engineered Pseudomonas putida
KT2440 in the absence of kanamycin.

| Trp | IAA titer (mg/L) | |
|---|---|---|
| (mg/L) | Pseudomonas putida/pGZ6 | Pseudomonas putida/pGZ5S |
| 0 | 106.9 ± 9.0 | 109.8 ± 0.8 |
| 800 | 552.9 ± 51.6 | 561.9 ± 25.5 |

It will be appreciated that various of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

### NON-PATENT CITATIONS

[1] Nicolopoulou-Stamati P, Maipas S, Kotampasi C, Stamatis P, Hens L: Chemical pesticides and human health: the urgent need for a new concept in agriculture. Frontiers in Public Health 2016, 4:148.
[2] Altomare C, Tringovska I: Beneficial soil microorganisms, an ecological alternative for soil fertility management. In: Genetics, Biofuels and Local Farming Systems. Edited by Lichtfouse E. Dordrecht: Springer Netherlands; 2011: 161-214.
[3] Lee K, Pankhurst C: Soil organisms and sustainable productivity. Soil Research 1992, 30(6):855-892.
[4] Fu S-F, Wei J-Y, Chen H-W, Liu Y-Y, Lu H-Y, Chou J-Y: Indole-3-acetic acid: A widespread physiological code in interactions of fungi with other organisms. Plant Signaling and Behavior 2015, 10(8):e1048052.
[5] Bunsangiam S, Thongpae N, Limtong S, Srisuk N: Large scale production of indole-3-acetic acid and evaluation of the inhibitory effect of indole-3-acetic acid on weed growth. Scientific Reports 2021, 11(1):13094.
[6] Kang B, Yang K-Y, Cho B, Han T, Kim I S, Lee M C, Anderson A, Kim Y: Production of indole-3-acetic acid in the plant-beneficial strain Pseudomonas chlororaphis O6 is negatively regulated by the global sensor kinase GacS. Current Microbiology 2006, 52:473-476.
[7] Aziz R K, Bartels D, Best A A, DeJongh M, Disz T, Edwards R A, Formsma K, Gerdes S, Glass E M, Kubal M et al: The RAST Server: Rapid Annotations using Subsystems Technology. BMC Genomics 2008, 9(1):75.
[8] Calderón C E, Ramos C, de Vicente A, Cazorla F M: Comparative genomic analysis of Pseudomonas chlororaphis PCL1606 reveals new insight into antifungal compounds involved in biocontrol. Molecular Plant-Microbe Interactions 2015, 28(3):249-260.
[9] Patten C L, Glick B R: Bacterial biosynthesis of indole-3-acetic acid. Canadian Journal of Microbiology 1996, 42(3):207-220.
[10] Martínez-García E, Nikel P I, Aparicio T, de Lorenzo V: Pseudomonas 2.0: genetic upgrading of P. putida KT2440 as an enhanced host for heterologous gene expression. Microbial Cell Factory 2014, 13:159.
[11] Nelson K E, Weinel C, Paulsen I T, Dodson R J, Hilbert H, Martins dos Santos V A, Fouts D E, Gill S R, Pop M, Holmes M et al: Complete genome sequence and comparative analysis of the metabolically versatile Pseudomonas putida KT2440. Environmental Microbiology 2002, 4(12):799-808.

What is claimed is:

1. A method of constructing microbial strains capable of efficiently producing indole-3-acetic acid. The said method comprises:
(a) obtaining indole-3-acetic acid biosynthetic genes through gene synthesis, PCR amplification, or digest of genomic DNA;
(b) obtaining a tryptophan transporter gene through gene synthesis, PCR amplification, or digest of genomic DNA;
(c) introducing the indole-3-acetic acid biosynthetic genes and tryptophan transporter gene into microbes;
(d) testing the ability of engineered strains to produce indole-3-acetic acid.

2. The method of claim 1 further comprises the use of a natural, recombinant, or synthesized tryptophan 2-monooxygenase such as Pc-IAA1 in microbes. The said Pc-IAA1 has an amino acid sequence shown in sequence listing.

3. The method of claim 1 further comprises the use of a natural, recombinant, or synthesized indoleacetamide hydrolase such as Pc-IAA2 in microbes. The said Pc-IAA2 has an amino acid sequence shown in sequence listing.

4. The method of claim 1 further comprises the use of a natural, recombinant, or synthesized tryptophan transporter such as Pc-IAA3 in microbes. The said Pc-IAA3 has an amino acid sequence shown in sequence listing.

5. The method of claim 1 further comprising an expression system of tryptophan transporter and indole-3-acetic acid biosynthetic genes with inducible or constitutive promoter in microbes. The expression can be achieved through an expression plasmid or insertion of the tryptophan transporter and indole-3-acetic acid biosynthetic genes into the genome of a microbial host.
